(19) United States
(12) Patent Application Publication
(10) Pub. No.: US 2024/0299314 A1
(43) Pub. Date: Sep. 12, 2024

(54) DRUG DELIVERING NANO DEVICES AND METHODS OF SYNTHESIZING AND CURING CANCERS USING THE SAME

(71) Applicants: TAN LE HOANG DOAN, HO CHI MINH (VN); DAT NGOC XUAN MAI, HO CHI MINH (VN)

(72) Inventors: TAN LE HOANG DOAN, HO CHI MINH (VN); DAT NGOC XUAN MAI, HO CHI MINH (VN)

(73) Assignees: VietNam National University Ho Chi Minh City, HO CHI MINH (VN); Center for Innovative Materials and Architectures, HO CHI MINH (VN)

(21) Appl. No.: 18/178,562

(22) Filed: Mar. 6, 2023

(51) Int. Cl. A61K 9/51; A61K 45/06; A61P 35/00

(52) U.S. Cl. CPC A61K 9/5192; A61K 9/5115; A61K 45/06; A61P 35/00; B82Y 5/00; B82Y 30/00; B82Y 40/00

(57) ABSTRACT

A method of synthesizing and a method of curing cancers using a nano device are disclosed, the nano device comprising: a framework comprising a structure formed by Si—O—Si covalent bonds and a plurality of mesopores configured to store drug cargos therein by disulfide covalent bonds; and an independent and separate chitosan layer, deposited directly on the framework. The chitosan layer is designed to coat and protect said framework layer from leaking the drug cargos in a first condition and to degrade so as to expose the drug cargos in a second condition. The framework releases the drug cargos by allowing a predetermined concentration of glutathione to break the disulfide (S—S) covalent bonds.

### FIELD OF THE INVENTION

The present invention relates generally to the field of nanomaterials. More specifically, the present invention relates to porous nanoparticles applicable to drug-delivering nanoparticles.

### BACKGROUND ART

Drug delivering nanoparticles have attracted much attention in biomedicine because they are dimensioned in nano scales that can be injected into the blood streams of a patient. These drug delivering nanoparticles move in the blood circulation to target sites. Upon arrival, they are functioned to deliver specific drugs to target sites such as cancer cells, tissues. However, in clinical applications, low degradation of nanoparticles have been discovered. Over time, the accumulation of the non-degraded nanoparticles may lead to toxicity. To solve these problems, new class of silica materials such as biodegradable periodic mesoporous organosilicas (BPMO) have been found degradable and biocompatible. In BPMOs, organic moieties are deposited in the porous silica framework. These degradable organic moieties are released when react with intracellular conditions as low pH, enzymic biomolecules, or redox. More particularly, high glutathione concentration in cancer cells triggers disulfide or tetrasulfide disintegration. Yet, due to the inherent long, flexible, and slightly soluble linkers, these BMPOs still have large particle sizes which adversely affect their drug delivering functions, which depends on conditions such as morphology, surface coating, agglomeration, route, during of exposure, uptake, cytotoxicity, and biodistribution.[1-10]

In 2014 X. Hu's research group successfully fabricated mesoporous silica nanomaterials (MSN) from tetraethoxysilane (TEOS) precursors and mounted pH-responsive chitosan. Chitosan is attached to MSNs by binding to (3-glycidyloxypropyl) trimethoxysilane (GTPMS) on the surface of MSNs. [Chitosan-Capped Mesoporous Silica Nanoparticles as pH-Responsive Nanocarriers for Controlled Drug Release, Chemistry—An Asian Journal, 9 (1), 319-327, (2014)]. The material's ability to load and release doxorubicin was evaluated in vitro. The results show the ability to respond effectively to the environment due to the chitosan component. As the pH value of the medium decreases, the drug release rate decreases accordingly. Furthermore, due to the biocompatibility of chitosan, the material has lower cytotoxicity compared to the non-chitosan-bound MSNs in the 3-(4,5-dimethylthiazol-2-yl)-2,5-diphenyltetrazolium bromide (MTT) experiment. In addition, the ability to kill MCF-7 breast cancer cells simultaneously increased with time and with the concentration of the drug-containing MSNs chitosan material. However, the MSN material has a large size of about 120 nm and is synthesized from inorganic silane precursors. In addition, X. Hu teaches that the chitosan must be bridged to the surface of MSNs by GTPMS. Thus, extra material (GTPMS) and extra steps are required in the X. Hu's chitosan-capped MSN. [11-19]

In 2017 J. Wu's research group successfully fabricated a multifunctional material based on a nano-core from iron oxide (Fe₃O₄) and a mesoporous silica core (Fe₃O₄@mSiO₂) as a carrier capable of controlling drug release by adjectives and pH response. [Synthesis and characterization of mesoporous magnetic nanocomposites wrapped with chitosan gatekeepers for pH-sensitive controlled release of doxorubicin, Materials Science and Engineering C, 70, 132-140 (2017)]. The material is magnetic and has a suitable size of less than 100 nm (<100 nm). Doxorubicin (DOX) was successfully loaded onto the drug carrier by electrostatic interaction with a loading amount of 29.3%. Chitosan is coated onto the drug carrier to prevent premature drug release of the material. At pH 4.0, 86.1% of DOX was released in 48 hours when the drug carrier was coated with chitosan. This chitosan-binding drug delivery material has effective tumor suppressor activity against the liver cancer cell line HepG2. Meanwhile, chitosan-free material beads (Fe₃O₄@mSiO₂) were not toxic to HepG2 cells. Although the material size is suitable for the application, the degradability of the material is not guaranteed due to the presence of durable inorganic bridges. [11-19]

In 2020 Y. Chen's research group synthesized materials as nanocarriers with structures including mesoporous silica nanoparticles (MSNs) as carriers, chitosan as collapsible nanovalves, and 1,8-naphthalimide fluorophore as a binding and fluorescence signal source. [Chitosan-Gated Fluorescent Mesoporous Silica Nanocarriers for Real-Time Monitoring Drug Release, Langmuir, 35 (24), 6749-6756, (2020)]. In the absence of glutathione (GSH)-induced drug release, the components of the nanocarriers are preserved. From there, the pores are closed and the sulfones prevent intramolecular charge transfer (ICT) resulting in no luminescence. Under the effect of GSH, the chitosan nanovalves were removed and the ICT properties were restored, thereby activating the drug release process and green fluorescence. The results demonstrate that changes in drug release as well as changes in fluorescence signal are affected by changes in GSH concentration. However, the MSN material with a large size of about 200 nm limits the ability of the material to disperse in vivo and is synthesized from inorganic silane precursors, causing limited biodegradation. [11-19]

Therefore, what is needed is a drug delivering nano device that has suitable sizes for biodistribution and agglomeration.

Furthermore, what is needed is a drug delivering nano device that is both biocompatible and biodegradable.

What is needed is drug delivering nano devices that are directly coated with independent and separate chitosan layer.

Finally, what is needed is a method of synthesizing a new drug delivering nano device that is simple and cost-effective. That is, what is needed is a method of synthesizing that requires fewer steps than the conventional ones.

The new drug delivering nano device and the method of synthesizing the same of the present invention solve the above-described problems and meet the market needs.

### SUMMARY OF THE INVENTION

Accordingly, an object of the present invention is to disclose a nano device comprising: a biodegradable periodic mesopore silicate nanoparticles (BPMO) framework comprising a cage-like structure formed by Si—O—Si covalent bonds and a plurality of mesopores configured to store drug cargos therein by disulfide (S—S) covalent bonds; and an independent and separate chitosan layer, deposited directly on the BPMO framework. The chitosan layer is designed to coat and protect the (BPMO) framework layer from leaking the drug cargos in a first condition and to degrade so as to expose the drug cargos in a second condition. The (BPMO) framework releases the drug cargos by allowing a predetermined concentration of glutathione to break the disulfide (S—S) covalent bonds without any participation of the chitosan layer.

Another object of the present invention is to provide a method for synthesizing a biodegradable and biocompatible drug delivering nano device (“nano device”) capable of delivering a specific drug cargo upon the occurring of an external stimuli, the process comprising the following steps: (a) synthesizing porous nanocarriers by creating a mixture of reaction medium including alkaline solution and a surfactant; (b) then adding dropwise silane precursors into the mixture in the following order: alkyl alkoxy silane or aryl alkoxy silane and then followed by sulfur alkoxy silane; condensing them at 80° C. for 2 hours; (d) depositing an independent and separate chitosan layer directly on the porous nanoparticles of step (a) by continually stirring the porous nanocarriers in the chitosan solution at room temperature for 48 hours; and (c) collecting the nano device by centrifuging the stirred mixture at a speed of 14,000 rpm to 20,000 rpm for 15 minutes to 45 minutes, and finally washing the powder product with water and ethanol.

Another object of the present invention is to provide a method for curing cancers using a biocompatible and biodegradable drug delivering nano device (“nano device”), comprising: (a) providing a biodegradable periodic mesopore silicate nanoparticles (BPMO) framework comprising a structure formed by Si—O—Si covalent bonds and a plurality of pores configured to store drug cargos therein by disulfide (S—S) covalent bonds; (b) adding drug cargos into the plurality of pores by mixing the drug cargos with the framework; (c) depositing an independent and separate chitosan layer directly on the BPMO framework; the chitosan layer is operable (1) to protect and to prevent the framework from leaking the drug cargos into a first condition and (2) to bio-degrade so as to expose the drug cargos in a second condition; the BPMO framework releases the drug cargos to the target by allowing a predetermined concentration of glutathione to break the disulfide (S—S) covalent bonds without the participation of the separate and independent chitosan layer; and (d) injecting the drug delivery nano-device into a patient.

Another object of the present invention is to provide a method of synthesizing a drug delivering nano device, comprising: (a) synthesizing a biodegradable periodic mesopore silicate nanoparticles (BPMO) framework having a structure formed by Si—O—Si covalent bonds and a plurality of pores configured to store drug cargos therein by disulfide (S—S) covalent bonds; and (b) coating the (BPMO) framework with a separate and independent layer of chitosan by depositing the separate and independent chitosan layer directly on the framework; the chitosan layer is operable to (1) coat and protect the (BPMO) framework layer from leaking the drug cargos in a first condition and (2) to degrade so as to expose said drug cargos in a second condition, and (3) to release the drug cargos by allowing a predetermined concentration of glutathione to break the disulfide (S—S) covalent bonds without any participation of the chitosan layer.

Another object of the present invention is to provide a biodegradable and biocompatible nano device for drug delivery that has zero premature control release, precise temporal control, high loading and encapsulation capacity, and efficient surface functionalization or capping.

Yet another object of the present invention is to provide a nano device for drug delivery that is easy to manufacture and cost-effective.

These and other advantages of the present invention will no doubt become obvious to those of ordinary skill in the art after having read the following detailed description of the preferred embodiments, which are illustrated in the various drawing figures.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 is a flow chart of a method for synthesizing a biodegradable and biocompatible nano device (“nano device”) coated with a separate and independent Chitosan layer designed to deliver a specific drug cargo in accordance with an exemplary aspect of the present invention;

FIG. 2 is a perspective diagram illustrating chemical reactions that form the nano device having an independent and separate chitosan coating layer in accordance with an exemplary embodiment of the present invention;

FIG. 3 presents the chemical reactions involved in the synthesis of the nano device in accordance with an exemplary embodiment of the present invention;

FIG. 4 is an illustration of a biodegradable and biocompatible nano device synthesized by the method described in FIG. 1 in accordance with an exemplary embodiment of the present invention;

FIG. 5 illustrates the drug cargo releasing process of the nano devices in accordance with an exemplary embodiment of the present invention;

FIG. 6 shows the drug cargo releasing reactions between the nano devices and glutathione (GSH) in accordance with an exemplary aspect of the present invention;

FIG. 7A-FIG. 7B show SEM and TEM images of the nano devices in accordance with an exemplary aspect of the present invention;

FIG. 8A-FIG. 8B show the N₂ adsorption-desorption isotherm of the nano devices in accordance with an exemplary aspect of the present invention;

FIG. 9 shows the biodegradability profile of the nano devices in accordance with an aspect of the present invention;

FIG. 10 presents effect of organic solvents on PTX loading capacity of the nano devices in accordance with an aspect of the present invention;

FIG. 11 shows the release profile of PTX using the nano device in accordance with an aspect of the present invention; and

FIG. 12A-FIG. 12C show a bar graph of the results of anticancer evaluation of PTX@NPs for fibroblast cell (L929), breast cancer cell (MCF-7) and viability ratio of cancer cell/normal cell in accordance with an aspect of the present invention.

### DETAILED DESCRIPTION OF THE INVENTION

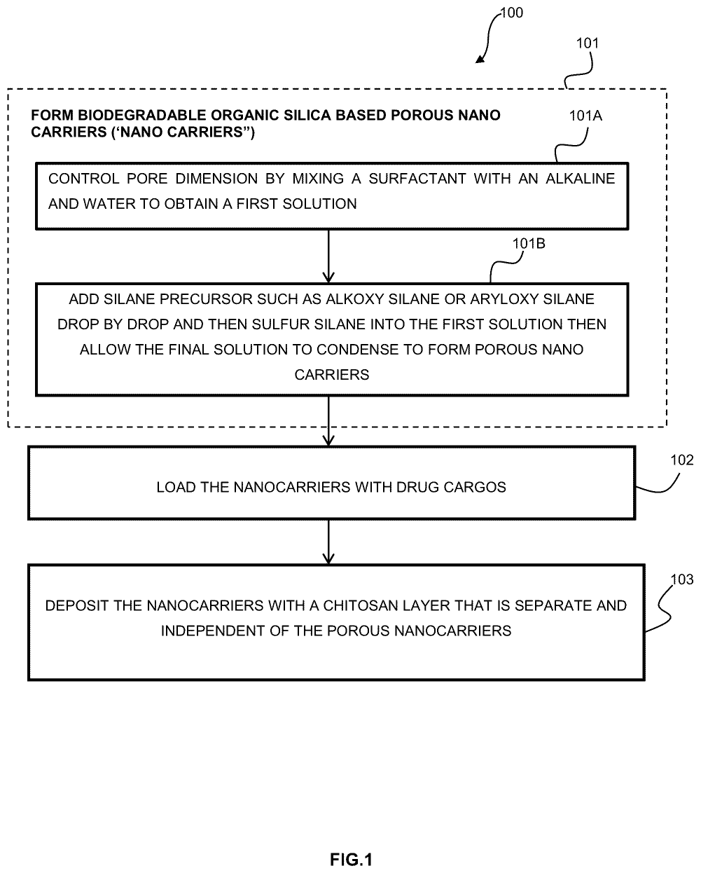

Referring now to FIG. 1, a flow chart of a method 100 for synthesizing a biocompatible and biodegradable nano device (“nano device”) configured to deliver selected drug cargos in accordance with an aspect of the present invention is illustrated. In particular aspects of the present invention, method 100 discloses the synthesis of biodegradable and biocompatible porous organic silica nanomaterials coated by a chitosan layer that satisfies the above-listed objectives of the present invention.

At step 101, porous nanocarriers are formed. The porous nanocarriers of step 101 are nanocarriers for loading drug cargos inside their mesopores. The nanocarriers include nanovalves and tunable pores. The mesopores are tunable using a predetermined reaction conditions between selected surfactant, alkaline, and water. These tunable mesopores have sizes from 2-10 nm. In many preferred aspects of the present invention, step 101 is realized by performing the following reactions in specifically disclosed sub-steps: At sub-step 101A, a first solution of alkaline and surfactant is formed. In various aspects of the present invention, the surfactant is cetyl trimethyl ammonium bromide or cetyl trimethyl ammonium chloride or similar. The alkaline solution used is sodium hydroxide (NaOH) or potassium hydroxide (KOH) solution. The mass ratio between surfactant and alkaline solution is between 3:1 and 9:1.

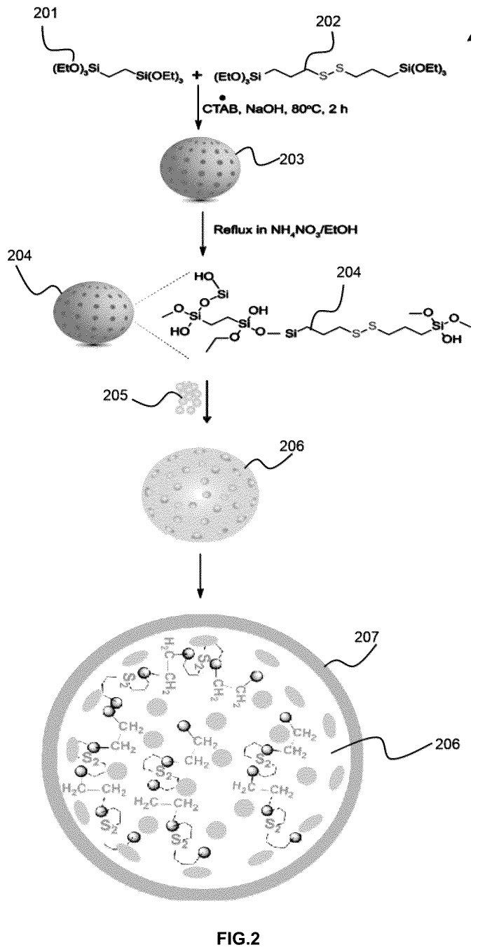

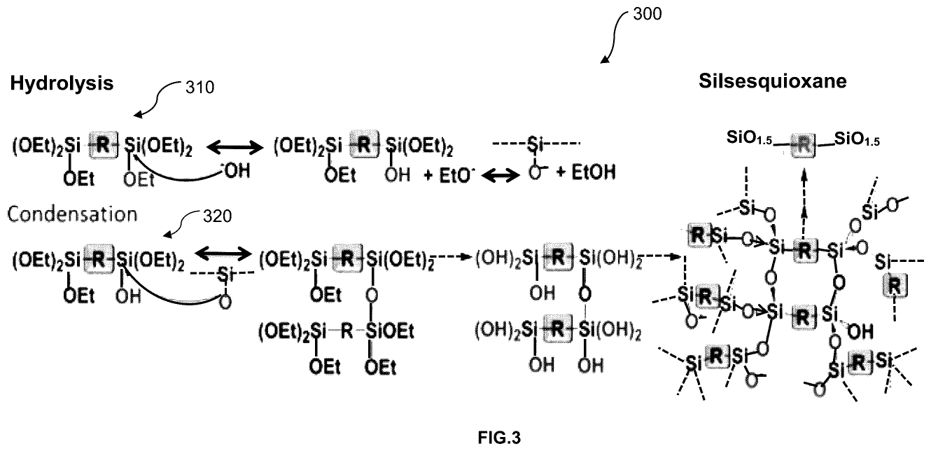

Sub-step 101A is followed by sub-step 101B, silane precursor such as alkyl alkoxy silane or aryl alkoxy silane is added dropwise to the first solution and then sulfur alkoxy silane is followed. In various aspects of the present invention, the mass ratio of surfactants and alkyl alkoxy silanes or aryl alkoxy silanes is between 2:1 to 3:1. Alkyl alkoxy silane is 1,2-Bis(triethoxysilyl)ethane or 1,2-Bis(triethoxysilyl)ethylene or similar; aryl alkoxy silane is 1,4-Bis(triethoxysilyl)benzene or 4,4′-Bis(triethoxysilyl)biphenyl or similar. Sulfur alkoxy silane is added from 5 minutes to 10 minutes; the mass ratio of the surfactant and sulfur alkoxy silane from 1:1 to 1:1.5; and sulfur alkoxy silane is bis[3(triethoxysilyl)propyl]disulfide, bis[3-(triethoxysilyl)propyl]tetrasulfide or similar. The mixture is thoroughly stirred. The reaction time is 2 hours at 80° C. Then the porous nanocarriers are collected by centrifugation for 30 to 45 minutes at a speed from 14,000 rpm to 20,000 rpm and washed at least twice with ethanol (CH₃CH₂OH). Then the surfactant is removed by refluxing overnight in a mixture of ammonium chloride (NH₄Cl) and ethanol (CH₃CH₂OH); then centrifuged for 30 to 45 minutes at a speed of 14,000 rpm to 20,000 rpm, rinsed with ethanol at least three times and collected biodegradable porous structure nanoparticles. Please refer to FIG. 2 and FIG. 3 for the illustration of step 101. After step 101, the nanocarriers are obtained that are ready to be loaded with specific drug cargos.

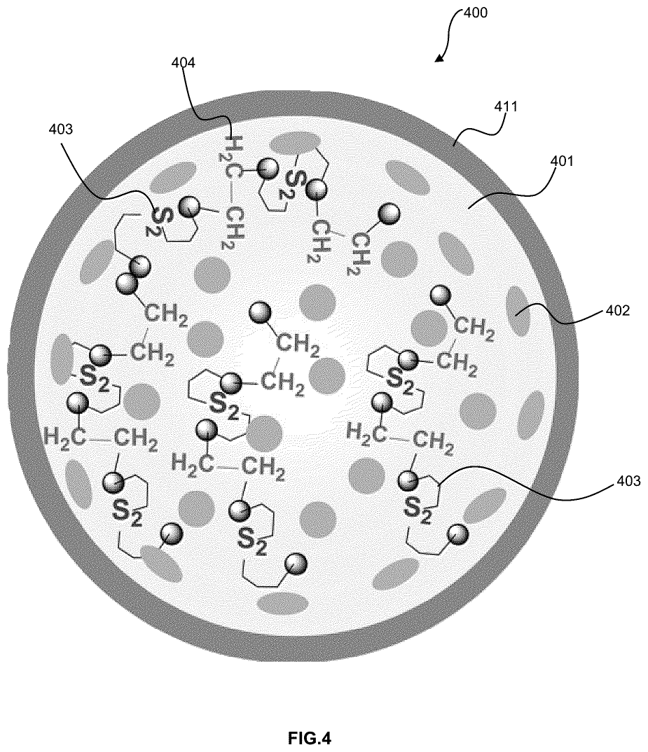

At step 102, drug cargos are loaded into the mesopores of the nanocarrier. Step 102 is realized by mixing the selected drug cargos with the nanocarriers. More specifically, the drug cargos are loaded onto the biodegradable material of the present invention by mixing the nanocarriers of the present invention with drug cargos at a predetermined dose using a magnetic stirrer. As a result, the nanocarriers of the present invention are loaded with the drug cargo inside the porous mesopores. In many aspects of the present invention, the drug cargo is paclitaxel (PTX) which is a hydrophobic anticancer drug. The loading capacity is up to 60 mg·g⁻¹. Experiments showed that a large amount of PTX was released at the early stage of the release process in the phosphate buffer saline solution containing reduced glutathione and then released slowly at a steady state. The effective cellular uptake of nanocarriers into malignant cells and normal cells was also demonstrated. PTX-loaded nano devices exhibit higher anticancer activity against cancer cells as compared to free PTX. Please see FIG. 4-FIG. 12.

Finally at step 103, the nanocarrier carrying drug cargos are coated with an independent and separate chitosan layer. Step 103 is realized by stirring the mixture of a chitosan solution and nanocarriers for 48 hours at room temperature. In other aspects of the present invention, biodegradable polymers are polysaccharides. In various preferred embodiments, the polysaccharide is chitosan, chitin, dextrin or their mixture. In one embodiment, the polysaccharide is chitosan. The chitosan solution is prepared by dissolving chitosan by dissolving 0.12 g of chitosan in 20 ml of 10% acetic acid solution (v/v). The pH of the solution is adjusted with 1 mole (1M) of NaOH solution until the pH of 6.0 is reached.

Step 103 is continued by collecting the nano device sample by centrifuging and washing with ethanol, water and ethanol solutions. The collection is conducted by centrifuging for a period of 30 minutes to 45 minutes at a speed from 14,000 rpm to 20,000 rpm, washing in turn with ethanol, water (H₂O), ethanol (EtOH) and collecting the chitosan-coated drug-delivering nano device. The drug is loaded onto the biodegradable material of the present invention by mixing the biodegradable material of the present invention with the drug solution at a predetermined dose using a magnetic stirrer so that the biodegradable material of the present invention loads the drug into its porous structure. With such reaction conditions performed by steps 101-103 above, the nano device obtained from method 100 above are a homogenous spherical morphology with an average size of 50 nm. Please see FIG. 4.

Referring now to FIG. 2, a perspective diagram 200 illustrating chemical reactions that form the nano devices with independent and separate chitosan coating in accordance with an exemplary embodiment of the present invention is illustrated. Diagram 200 illustrates the chemical reactions and steps that realize method 100 discussed above.

The dimension of the mesopores is designed by the reactions between surfactant, alkali, and water. Surfactants such as Hexadecyltrimenhylammonium bromide or centrimonium bromide (CTAB —[C₁₆H₃₃)N(CH₃)₃]Br) is mixed with alkaline such as NaOH or KOH to obtain the first solution. Under the preferred reaction condition, this reaction is carried out at 80° C. for 2 hours. The surfactant modules of CTAB self-aggregate at alkaline pH to form micelles. Then the cage-like structure is formed by adding 1,2-Bis(triethoxysilyl)ethane 201 drop by drop into the first solution and followed by sulfur alkoxy silane such as bis(triethoxysilyl) disulfide 202 also drop-by-drop. This reaction causes micellar packing to start through the electrostatic interaction between positively charged N⁺(CH₃)₃ of CTAB and negatively charged silanes (Si—O⁻). This leads to the formation of the ordered architecture of silica 203. The silica precursors overlap at the polar head of the surfactant micelles by forming a wall around them. The structure-directing agent CTAB is removed by refluxing overnight in a mixture including 0.3 g of NH₄Cl and 50 ml of ethanol. The refluxed product is then centrifuged for 30 min at 14,000 rpm and washed three times with ethanol (EtOH) and obtain the nanocarriers 204 which are biodegradable periodic mesoporous organosilica nanomaterials (BPMO) containing 1,2-Bis(triethoxysilyl)ethane and bis[3-(triethoxysilyl)propyl]disulfide to expose channels of mesopores. Finally, the product is dried at 80° C. Nanocarriers (BPMO) 204 is a silsesquioxane that has a cage-like structure with Si—O—Si linkages.

Next, drug cargos 205 are loaded to nanocarriers 204 to form nanocarrier 206. The loading of drug cargos 205 may be realized by mixing so that drug cargos 204 are linked with Si—O—Si covalent bonds inside nanocarriers 204 that has the porous structure of BMPO. In many aspects of the present invention, drug cargos 205 is paclitaxel (PTX) which is a hydrophobic anticancer drug. The loading capacity of nanocarriers 204 is up to 600 mg·g⁻¹.

Finally, a separate and independent chitosan solution 207 and nanocarrier 206 are mixed together at room temperature for 48 hours. Chitosan solution 207 is prepared by dissolving 0.12 g of chitosan in 20 mL of 10% acetic acid solution (v/v). The pH of the solution is adjusted with 1 mole (1M) of NaOH solution until the pH of 6.0 is reached.

Next referring to FIG. 3, chemical reactions 300 to form silsesquioxane structure in accordance with an exemplary embodiment of the present invention are illustrated. It is noted that FIG. 3 only illustrates chemical reactions 300 that form the cage-like nanocarriers without the loading of drug cargos 205 and the chitosan layer 207. Also refer back to FIG. 2, 1,2-Bis(triethoxysilyl)ethane 201 is allowed to react to sulfur alkoxy silane such as bis(triethoxysilyl) disulfide 202 with the catalyst of CTAB, alkaline (NaOH and KOH), and water to form a spherical silica structure 203 composed of silsesquioxane (SiO_{3/2}—R—SiO_{3/2}). Silsesquioxane provides cage-like framework for spherical silica structure 203 that is formed over the micellar formation of CTAB. As a reminder that CTAB and the above specified reaction condition determine the pore size while silsesquioxane determines the morphology of spherical silica structure 203. More particularly, reactions 300 include a hydrolysis stage 310 of either alkyl alkoxy silane or aryl alkoxy silane which is denoted as R. In the hydrolysis reaction 310, (OEt)₂Si(OEt)-R—Si(OEt)₂+H₂O→(OEt)₂Si—R—SiOH(OEt)₂ where OEt is ethoxy functional group with chemical structure —O—CH₂—CH₃, and EtOH is ethanol. The OH⁻ cations replace the OEt⁻ at the Si-bonds. Free OEt⁻ cations are combined with an H⁺ anions of water to form ethanol (EtOH) and SiO⁻. Please refer to FIG. 3.

Next, in the condensation reaction 320, (OEt)₂Si—R—Si(OEt)₂ is condensed with SiO⁻ many times to form silsesquioxane cage-like structure that includes the Si—O—Si covalent bonds. In many aspects of the present invention, reactions 300 form nano devices 206 that are biodegradable periodic mesoporous organosilica (BPMO) material. BPMO material is a porous material selected from sulfur alkoxy silane like disulfide (S—S) or tetrasulfide (S—S—S—S) bridges; alkyl alkoxy silane or aryl alkoxy silane groups like ethane (C—C), ethylene (C═C), phenylene (—C₆H₄—) or biphenyl (—(C₆H₄)₂—) bridges. A coating layer 207 is a separate biodegradable polymer depositing directly on the surface of nanocarriers 206. Wherein biodegradable polymers are polysaccharides; and in one embodiment, the polysaccharide is chitosan, chitin, dextrin or their mixture. Wherein, sulfur alkoxy silane source is bis[3-(triethoxysilyl)propyl]tetrasulfide or bis[3-(triethoxysilyl)propyl]disulfide; the alkyl alkoxy silane or aryl alkoxy silane source is 1,2-Bis(triethoxysilyl)ethane, 1,2-Bis(triethoxysilyl)ethylene, 1,4-Bis(triethoxysilyl)benzene, or 4,4′-Bis(triethoxysilyl)biphenyl.

Referring now to FIG. 4, a diagram of a biodegradable and biocompatible nano device 400 synthesized by the method described in FIG. 1 and FIG. 2 in accordance with an exemplary embodiment of the present invention is illustrated. Nano device 400 includes a Silsesquioxane framework 401 which is described in FIG. 3. Silsesquioxane framework 401 covers mesopores 402 formed by CTAB and NaOH catalyst first solution described above in FIG. 2. Each mesopore 402 has a size about 1-4 nm. Silsesquioxane framework 401 covers mesopores 402 formed by CTAB and NaOH catalysts. First solution described above in FIG. 2. Each mesorepore 402 has a size about 1-4 nm. Silsesquioxane framework 401 has a surface area in the range of 50 nm to 100 nm. The average size of framework 401 is about 60-80 nm. The specific surface area is 150-900 m²/g. Si—O—Si bonds 403 from silsesquioxane framework 401 and C—C bonds 404. A separate and independent chitosan layer 411 protects the entire nanocarrier structure.

Continuing with FIG. 4, the chitosan-coated biodegradable periodic mesoporous organosilica material of nano device (“nano device”) 400 with a uniform spherical size is analyzed for zeta potential. The zeta potential of nano device 400 is from 3.67 to 21.97 mV depending on chitosan concentration. On the other hand, nano device 400 have a zeta potential of −18.73 mV. The increase in zeta potential value demonstrated that chitosan is successfully attached to nano device 400. In various preferred embodiments of the present invention, chitosan layer 411 is deposited directly on the surface of the material. The sole function of chitosan layer 411 is to protect silsesquioxane framework 401. Chitosan layer 411 of the present invention does not participate in the therapeutic functions of nano device 400. Chitosan layer 411 reacts to acidic pH in the range of pH 4.0 to pH 5.5 under the reduced pH conditions created high glutathione concentrations (10 mM). This leads to the release of drug cargoes 205 at the target sites.

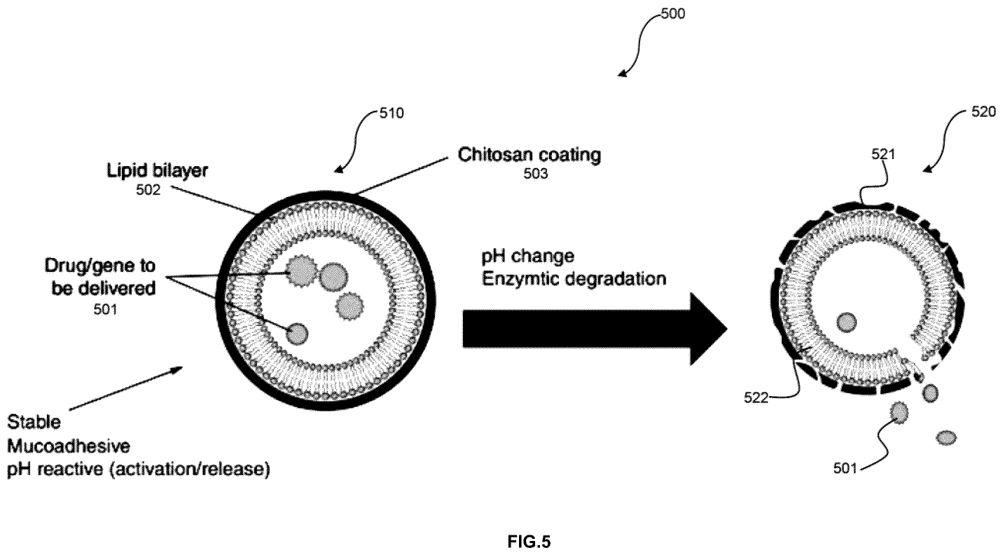

Next referring to FIG. 5, a perspective diagram 500 of the drug delivering reactions of the nano device in accordance with an exemplary aspect of the present invention is illustrated. A nano device 510 includes drug cargos 501, an organosilica framework 502 that is BPMO material 204, and an independent and separate chitosan layer 503. In the present invention, as alluded to above, independent and separate chitosan layer 503 is designed to protect drug cargos 501 and organosilica framework 502 by the following mechanism: (a) act as a valve to release drug cargo 501 to the target sites; and (b) does not participate in the curing of diseases because it is designed to biodegrade at the target sites. In neutral conditions (pH=7.4) when nano device 510 is en route to the target cells, chitosan layer 503 protects drug cargos 501 and organosilica framework 502. Independent and separate chitosan layer 504 helps achieving zero premature controlled release and precise temporal control (no leakage). At the target site, when pH changes and under enzymatic degradation, chitosan layer 521 starts to bio-degrading, acting as nano valves—prerequisites for the release drug cargos 501. Next the release of drug cargos 501 happens when the pH of surrounding environment of the target sites further reduces to between 5.5 and 4.0, exposing an organosilica framework 502 to the external environment filled with glutathione (GSH). Organosilica framework 522 degrades, cleaves, and releases drug cargos 501. During this cleavage and drug cargos release, nano device 510 becomes disintegrating nano device 520.

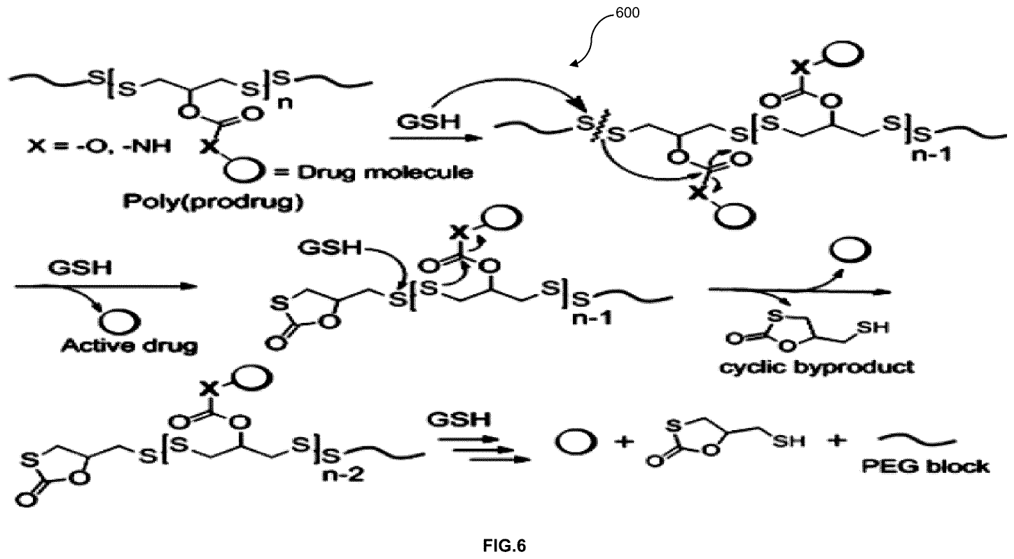

Next, FIG. 6 shows redox reactions of glutathione (GSH) that release the drug cargos to the target sites in accordance with an exemplary aspect of the present invention. The present invention takes advantages of the reducing behavior of glutathione—an antioxidant abundant in mammalian cells—to design nano device 510 that releases drug cargos 501 to the target sites such as cancer tissues. The S—S covalent bonds conjugated with drug cargo 501 (e.g, paclitaxel (PTX)) are broken by the reducing pathways of the thiol group in the cysteine of glutathione. Referring back to FIG. 5, after chitosan layer 521 degraded to begin the release process of drug cargos 501, glutathione (GSH) functions to neutralize the oxidative stresses caused by the invading nano device 510. Reducing agents H⁺ and electrons from GSH are released from the thiol groups (R—SH). This is because glutathione is acidic (α-aminocarboxylic acid) and its pH is very low. The S—S covalent bonds are cut and disulfide (S—S) exchanges occur that break those of organosilica framework 522. The end result is the release of drug cargo 501 and thiol group (SH—R).

### EXPERIMENTS

The following experiments were performed to support the enablement and sufficient disclosure of the present invention.

### Materials and Methods

### Chemicals and Reagents

Hexadecyltrimethylammonium bromide (CTAB) was received from Acros Organics Company. 1,2-Bis(triethoxysilyl)ethane, bis(triethoxysilyl) disulfide, Fluorescein isothiocyanate isomer I (FITC), (3-Aminopropyl) triethoxysilane (APTES), dichloromethane (DCM), dimethyl sulfoxide (DMSO), ethanol, and paclitaxel (PTX) were obtained from Sigma-Aldrich. Ammonium nitrate was obtained from Wako Pure Chemical Corporation (Japan). All reagents are used without further purification. Milli-Q water was used for all preparation of solutions.

### Nanocarriers Synthesis

The synthesis of disulfide-based nano devices was based on our previous procedure by changing the organosilica precursor in the framework [9]. Firstly, a mixture of CTAB (250 mg, 0.69 mmol), water (120 mL), and sodium hydroxide (NaOH) 1 M (800 μL) was continuously stirred at the temperature of 80° C. 1,2-bis(triethoxysilyl)ethane (300 μL, 0.88 mmol) was added dropwise to the above solution followed by adding dropwise bis(triethoxysilyl) disulfide (100 μL, 0.2 mmol). The reaction was maintained at 80° C. for 2 hours under continuous stirring. The synthesized sample was separated by centrifugation at 16,000 rpm for 30 min and then washed with ethanol. To remove residual CTAB inside the porous structures, the synthesized nano devices were refluxed overnight in an ammonium nitrate alcoholic solution. The sample was collected and washed again several times with ethanol and water. Finally, the obtained nano devices were activated under vacuum at 80° C. for 24 hours. Fluorescent was labeled to nano devices via post-synthesis with FITC-APTES. In particularly, stock of FITC was prepared by mixing of FITC (5 mg), EtOH (5 mL), and APTES (20 μL). After that, the mixture of nano devices (20 mg), EtOH (20 mL), and FITC stock (1 mL) was stirred for 24 h at 25° C. in dark condition. FITC-labeled nano devices were collected by centrifugation and washed several times with DI water.

### Characterization

Scanning electron microscope and transmission electron microscopy images were observed by using a S4800 Hitachi and a JEOL JEM-2100 F equipment, respectively. Thermal gravimetric analysis (TGA) was operated on a TA Instruments Q-500 thermal gravimetric analyzer under continuous airflow from room temperature to 800° C. with a heating rate of 5° C. min⁻¹. The N₂ adsorption isotherm was observed by a Quantachrome Autosorb iQ2 at 77 K. Ultrahigh-purity-grade N₂ and He (99.999% purity) were used throughout the operation. Fourier transform infrared (FT-IR) spectra were collected by a Bruker Vertex 70 FT-IR spectrometer. X-ray photoelectron spectroscopy (XPS, K-alpha, Thermo) was applied for evaluating the chemical states of the nano devices.

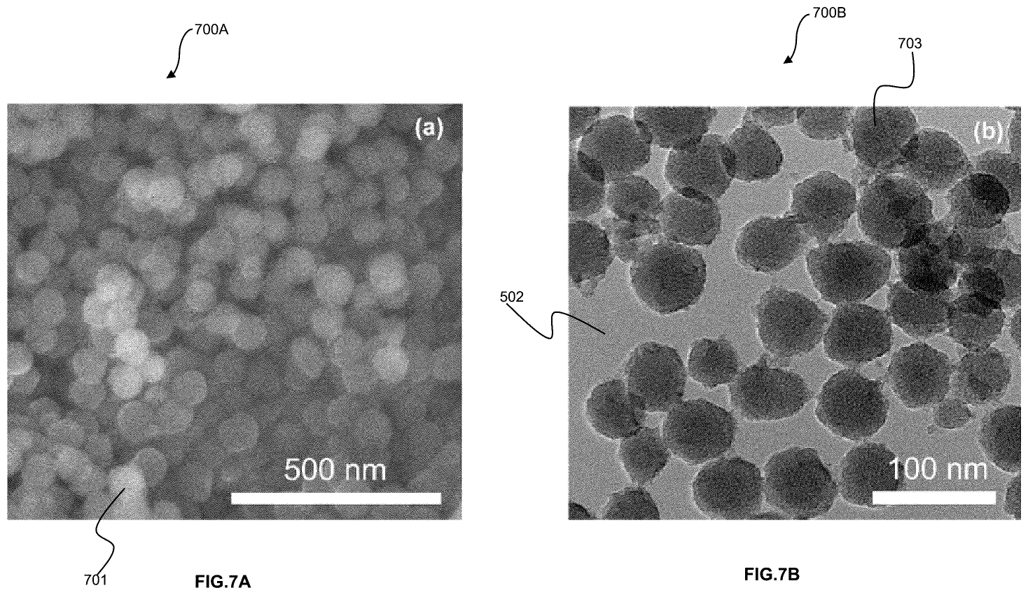

Referring now to FIG. 7A, a Scanning Electron Microscope (SEM) image 700A of nano device 400 in accordance with an exemplary embodiment of the present invention is illustrated. In the SEM experiment, electron beams are scanning across the surface of the sample. As such, nano device 701 obtained from method 100 are showed to have homogenous spherical particles with an average size of 50 nm. In FIG. 7B, a Transmission Electron Microscope (TEM) image 700B shows the homogeneous spherical particles of nano device 400 with an average diameter of 50 nm. In TEM, the electron beams are transmitted through the sample. Thus, nano carriers 703 and void spaces 702 are shown.

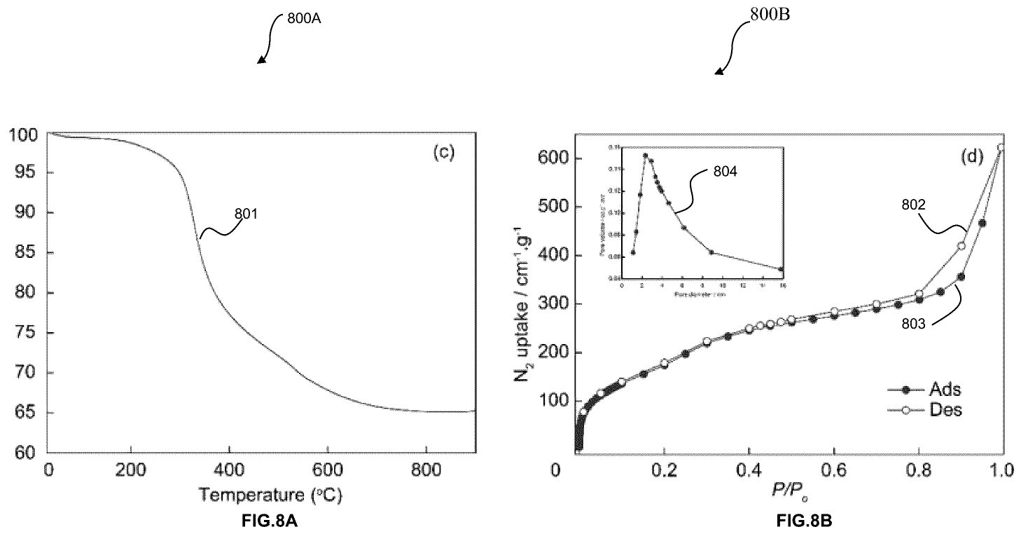

In FIG. 8A, a graph 800A of thermogravimetric analysis (TGA) of the nano device obtained from method 100 above is shown. TGA is a method of thermal analysis in which the mass of nano device 400 is measured over time as the temperature changes. In the present invention, TGA graph 800A is used to evaluate thermal stability of nano device 400. The vertical axis is the percentage mass and the horizontal axis is temperature in Celsius. TGA measurement of nano device 400 is represented by graph 801. Graph 801 shows that in temperatures below 200° C. there is no significant change in mass of nano device 400. That is under 200° C. there is negligible mass loss corresponding to little or no slope in curve 801. Curve 801 also gives the upper limit temperature of nano device 400. Beyond 200° C., nano device 400 begins to degrade. The main weight loss of approximately 35% is attributed to the organic fractions of S—S and C—C linkers in the framework. Nano device 400 is decomposed at approximately 350° C. Furthermore, the insignificant weight loss at below 100° C. is corresponded to ethanol aqueous solution about 1%.

In FIG. 8B, a graph 800B of N₂ adsorption-desorption isotherms of nano device of the present invention. N₂ adsorption-desorption isotherms measure pore structure analysis of a porous material over a wide range of relative pressure (P/P₀). A graph 802 characterizes N₂ adsorption and a graph 803 is N₂ desorption of nano device 400 measured at a constant temperature (isotherms) at 77 K (−196° C.). From graph 802 and graph 803, inflection points (I-point) are known. Identical inversion point (I-point, Inversion or Inflection point), which corresponds exactly to the monolayer volume Vₘ. As a result, the value of specific surface area S is determined with unique precision via the trivial relationship S (m² g⁻¹)=4.356Vₘ(cm³ g⁻¹) without any use of the classic BET treatment. The derivatives

$$\frac{dV}{d ( P / {P}_{0} )} vs . \frac{P}{{P}_{0}} and \frac{dV [ 1 - \frac{P}{{P}_{0}} ]}{d ( \frac{P}{{P}_{0}} )} vs . \frac{P}{{P}_{0}} ,$$

exhibit maxima exactly at the same pressure

$$\frac{P}{{P}_{0}} ,$$

while the second order derivative

$$\frac{{d}^{2} V}{{d}^{2} [ \frac{P}{{P}_{0}} ]} vs . \frac{P}{{P}_{0}} ,$$

exhibits a minimum exactly at the same pressure

$$\frac{P}{{P}_{0}}$$

with the I-point. As a consequence, the specific surface area of solids can be determined just from experimental raw

$$\frac{P}{{P}_{0}} , V$$

data without any use, neither of linear BET treatment nor the Inversion or Inflection type graphs. Isotherm graph 802 is of type IV, which is specific for mesoporous structures. In addition, the calculated Brunauer-Emmettt-Teller (BET) surface is 701.90 m² g⁻¹ with a pore volume of 1.181 cm³·g⁻¹. The pore diameter calculated from the Barrett-Joyner-Halenda (BJH) analysis as shown in a graph 604 is approximately 2.50 nm, which corresponds to the property of mesoporous materials.

### Drug Loading and Release Experiments

PTX was loaded into the porous nano devices by the adsorption equilibrium method [20, 21]. Briefly, 1 mg nano devices were added into 1 mL PTX solution (1 mg·mL⁻¹) and stirred for 24 h at room temperature. The PTX solutions were prepared in different solvents to optimize the drug loading capacity. The supernatant was separated by centrifugation and stored for high-performance liquid chromatography (HPLC). HPLC analyses were recorded on Agilent Technologies 1200 Series with ZORBAX SB-C18 column (Agilent, 5 μm, 4.6×250 mm). The mobile phase was water: acetonitrile (50:50 v/v). The separation was carried out at 30° C. The detection wavelength was 227 nm and a flow rate of 1.0 mL·min⁻¹ was employed. A sample volume of 20 μL was injected. The loading capacity of nano device was calculated based on the following formula: Loading capacity

$$( mg \cdot {g}^{- 1} ) = \frac{{m}_{o} - {m}_{r}}{{m}_{NPS}}$$

where mₒ (mg) is the initial amount of PTX used; mᵣ (mg) is the residual amount of PTX in the loading solution; m_{NPs} (g) is the number of devices used in the loading experiment. A dialysis bag diffusion technique was used for examining drug release behaviors [14]. Briefly, PTX@NPs were dispersed in 1 mL of release solution (DMSO: PBS 1:9 v/v) and transferred to a dialysis bag (molecular weight cut off=14 kDa) and shaken in 5 mL of release solution at 37° C. At the predetermined time intervals, 200 μL of the solution was withdrawn and the same amount of fresh solution was added into the system. The released PTX content from PTX@NPs was determined by HPLC analysis. HPLC analyses were recorded on Agilent Technologies 1200 Series with ZORBAX SB-C18 column (Agilent, 5 μm, 4.6×250 mm). The mobile phase was water:acetonitrile (50:50 v/v). The separation was carried out at 30° C. The detection wavelength was 227 nm and a flow rate of 1.0 mL·min⁻¹ was employed. A sample volume of 20 μL was injected. Free PTX was used as a control to compare with PTX@NPs.

### Cellular Uptake

In order to investigate the cellular uptake of nano devices, they are first fluorescently labeled. Fluorescein isothiocyanate (FITC) detects the presence of nano devices by measuring the fluorescence intensity of labeled nanoparticles. L929 and MCF-7 cells were cultured and incubated for 24 h in a 96-well plate for cellular growth. After 0, 0.5, 1, 2, 4, 6 and 24 hours following the introduction of FITC-labeled nano devices, the cellular uptake is examined. After that, the culture medium was discarded and twice cleaned with PBS to remove the nano devices that have not yet entered the cells. DMSO is added to dissolve the nano devices and disrupt the cell membranes. The fluorescence intensity with excitation of 495 nm and emission of 519 nm was used to evaluate the uptake of nano devices by using a fluorescent microplate reader (Thermo Scientific™ Varioskan™, USA). Cellular uptake

$$( \% ) = \frac{{F}_{t e s t} - {F}_{b l a n k 1}}{{F}_{c o n t r o l - {F}_{b l a n k 2}}} \times 100 \%$$

Where F_{control} is the fluorescence intensity of the control (samples and DMSO). Fₜₑₛₜ is the fluorescence intensity in the presence of the samples at each time (cells, samples and DMSO solvent). F_{blank1} is the fluorescence intensity of the blank1 (cells and DMSO solvent) and F_{blank2} is the fluorescence intensity of the blank2 (water and DMSO).

### In Vitro Cytotoxicity Assay

Anticancer efficacy and cytotoxicity of PTX and PTX@NPs against cancer and normal cells were investigated by using MTT (3-[4,5-dimethylthiazol-2-yl]-2,5 diphenyl tetrazolium bromide) assay. The main mechanism of this method is the conversion of MTT into formazan crystals by mitochondrial activity in the living cells. Breast cancer cell line (MCF-7) and fibroblast cell (L929) were used and cultured in Dulbecco's modified Eagle's medium (Sigma-Aldrich) with 10% fetal bovine serum (Sigma-Aldrich) and 1% antibiotics (penicillin, streptomycin, and neomycin; Gibco) at 5% CO2 and 37° C. To study the effects of PTX and PTX@NPs on the viability of the cell, MCF-7 and L929 cell lines were seeded on a 96-well plate (5×103 cells/well) and incubated for 24 hours. The tested samples including PTX, PTX@NPs, and nano devices were added to the cell culture media and incubated for another 24 hours, followed by adding MTT (Roche Diagnostics, Tokyo, Japan) to each well and incubating them for another 4 h. The formazan crystals are dissolved by the addition of dimethyl sulfoxide and the absorbance of each well is measured at 540 nm using a microplate reader (Thermo Scientific™ Varioskan™, USA). Cell viability

$$( \% ) = \frac{{A}_{t e s t} - {A}_{b l a n k}}{{A}_{c o n t - {A}_{b l a n k}}} x \times 100 \%$$

Where A_{cont} is the absorbance of the control; Aₜₑₛₜ is the absorbance in the presence of the samples and A_{blank} is the absorbance of the blank (MTT and DMSO solvent). To directly observe the viability of cancer cells after drug treatment, fluorescein diacetate (FDA, Sigma-Aldrich Co. LLC) and propidium iodide (PI, Sigma-Aldrich Co. LLC) were used to stain viable cells and dead cells, respectively. After 24 hours treated with PTX and PTX@NPs, cells were incubated at 37° C. with FDA and PI for 10 min in a dark incubator. Then, the medium was removed and washed the samples 3 times with PBS. The images were taken by using a fluorescence microscope (Nikon, Osaka, Japan) at 40× magnification.

### Results and Discussion

The biodegradable disulfide-based mesoporous silica nano devices were synthesized via sol-gel reaction using ethane- and disulfide-containing organosilica precursors (please refer to FIG. 2). The porous structure of materials was formed under the catalyst of CTAB template and alkaline catalyst. The effect of various reaction conditions, including starting precursor ratio, alkaline concentrations, and temperature, on obtained nanoparticles were evaluated. Various nanoparticles are collected with different shapes and dispersion properties (FIG. 7A-FIG. 7B). The homogenous spherical particles are obtained by the starting precursor ratio of 3:1 (v/v) at 80° C. (FIG. 7A-FIG. 7B).

### Characterization

Scanning electron microscopy and transmission electron microscopy (TEM) were conducted to evaluate the morphology of nano devices. The homogeneous spherical particles with an average size of 50 nm were observed in SEM and TEM (please refer to FIG. 7A-FIG. 7B). Thermogravimetric analysis (TGA) was applied to confirm the presence of organic components of the silica particles. As can be seen from the TGA curve, the main weight loss of approximately 35% was attributed to the organic fractions of S—S and C—C linkers in the framework. The materials were decomposed at approximately 350° C. Furthermore, the insignificant weight loss at below 100° C. is corresponded to ethanol aqueous solution (about 1%). That change indicated the successful activation of materials (see FIG. 8A). On the other hand, the residual of PTX@NPs sample was approximately 20% lower than that of NPs sample. This is corresponding to the PTX content which is loaded in the nano devices). To evaluate the porous properties of nano devices, nitrogen adsorption-desorption isotherm was conducted. The isotherm of nano devices was of type IV, which is specific for mesoporous structures (see FIG. 8B). In addition, the calculated Brunauer Emmett-Teller (BET) surface was 701.90 m²·g⁻¹ with a pore volume of 1.181 cm³·g⁻¹. The pore diameter calculated from the Barrett-JoynerHalenda (BJH) analysis was approximately 2.50 nm, which corresponds to the property of mesoporous materials. The obtained porosity of NPs was appropriate for loading small paclitaxel molecules in their structures. It can be seen that the BET specific surface area obviously decreased after loading PTX into porous structure of nano devices, approximately 435.85 m²·g⁻¹. These obtained results demonstrated the success of PTX loading.

The chemical compositions of nano devices and two precursors were preliminarily determined by FT-IR with the range from 4,000 to 600 cm⁻¹. The C—H stretching vibrations of —CH₂—CH— groups were observed by peaks from 2800 to 3,000 cm⁻¹ for all spectra. It is worth noting that the high intensity peaks around 1,156 cm⁻¹ indicated the formation of Si—O—Si in the silica framework of nano devices while absent in the spectra of the two precursors. In addition, the peaks around 686 and 691 cm⁻¹ can be assigned to C—S stretches in the spectra of the S—S precursors and nano devices, respectively. These observations demonstrated the successful incorporation of the two organosilica precursors into the silica framework. Moreover, the obtained specific characteristic adsorption peaks of PTX at 1024 cm⁻¹ and 709 cm⁻¹ in the PTX@NPs spectra indicated the presence of PTX in the nanoparticles. XPS was performed to further determine the chemical composition in the materials. The XPS wide-range spectra of NPs. The five peaks of the XPS spectra at 532.84, 285.45, 164.25, and 102.93 eV represent the binding energies for O 1 s, C 1 s, S 2p and Si 2p, respectively. It is clear that C 1 s provides four different peaks. Two first component at 286.0 eV and 284.5 eV corresponds to the C—O bonds and C—C bonds, respectively. It is interesting that the other two peaks observed at 289.9 eV and 287.8 eV are associated with C—O bonds. The presence of disulfide-linker is confirmed through the deconvolution of the S 2p spectrum. It can be seen that two different peaks attributed to binding energy for Sp3/2 at 163.3 eV and Sp1/2 at 164.5 eV indicated the successful condensation of disulfide in the silica framework. XPS Si 2p of nanoparticles demonstrated the formation of Si—O—Si framework due to the obtained peak corresponded to the binding energy for Si—O—Si at 103.7 eV. In addition, the SiO₂₋ₓ component was observed at 102.4 eV. Two components at 532.3 eV and 532.9 eV in the O 1 s spectrum respectively designated to C—Si and C—O in the structure. In addition, the peak detected at 531.1 eV is associated with C—O bond that correlated with the result in the C 1 s spectrum. As expected, the successful incorporation of disulfide linker in the porous structures of nano devices is further demonstrated via these XPS results.

### Degradation

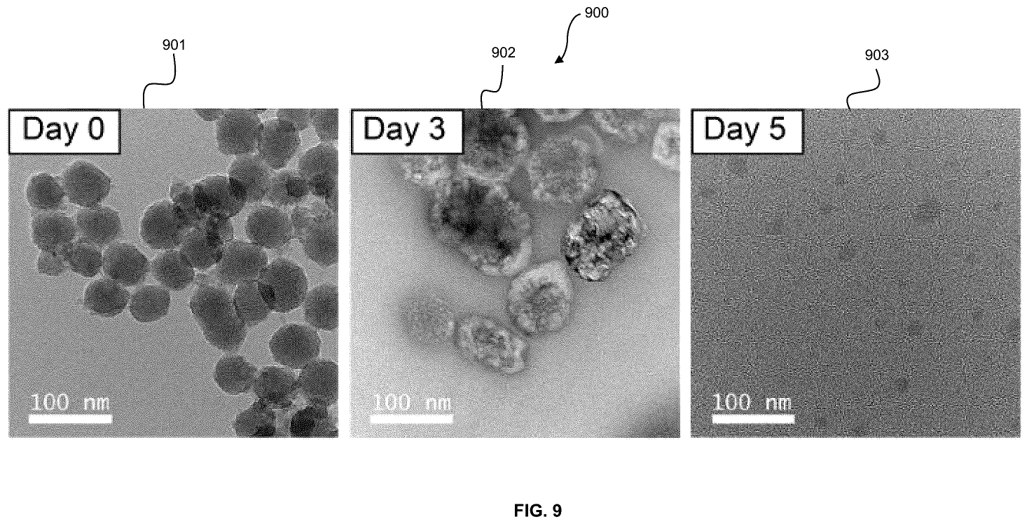

Now referring to FIG. 9, TEM images 900 showing the biodegradability of the nano devices of the present invention were obtained. The biodegradability of nano devices was investigated to confirm the successful incorporation of disulfide organic linkers into the particle structure. To evaluate the degradation profile, the nano devices were immersed in phosphate buffer saline solution (PBS pH 7.4) with the addition of glutathione (10 mM GSH). After each time interval, samples were evaluated by TEM. An image 901 shows the morphology of nano devices after degradation. An image 902 shows after 3 days incubated with PBS/GSH, the 50 nm spherical nano devices appeared to spread out, which is resulted from the degradation of linkers. An image 903 shows that the nano devices degraded continuously into smaller fractions of about 5 nm within 5 days, which can be easily excreted into urine and then cleared from the body. Furthermore, nano devices in this work were decomposed faster than most reported materials. It is completely degraded after 5 days while ethane-containing tetrasulfide-based nano devices took 7 days [5] and phenylene-containing tetrasulfide nano devices were completely decomposed after 2 weeks [25]. It is clear that the disulfide linkers reduce significantly the degradation time of materials than that of materials with tetrasulfide linkers.

### Drug Loading and Release

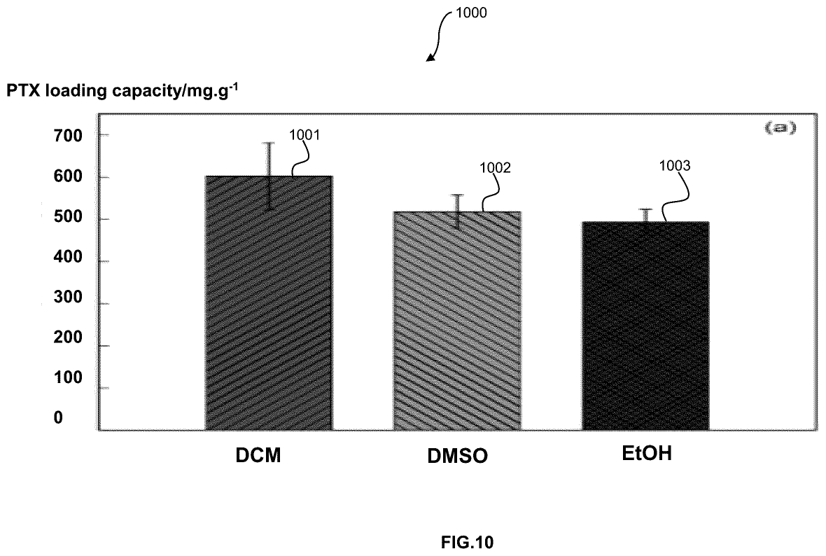

Now referring to FIG. 10, a bar graph 1000 showing the PTX loading capacity/mg·g⁻¹ is shown. The loading of PTX into the porous structure of nano devices was conducted in various solvents including dichloromethane (DCM) 1001, dimethyl sulfoxide (DMSO) 1002, and ethanol (EtOH) 1003. As shown in FIG. 10, the calculated loading capacity of nano devices toward PTX of nano devices was highest when loaded in up to 602.15 mg·g⁻¹ in the DCM solvent 1001. On the other hand, the capacity was lower when the loadings of DMSO 1002 and EtOH 1003 were 517.86 and 493.67 mg·g⁻¹ respectively. The significant enhanced capacity obtained in DCM 1001 could be due to its polarity being lower compared to other solutions and then less competing with PTX molecule in absorption into nano devices [14, 26]. Hence, changing the loading solvent could adjust the drug loading capacity.

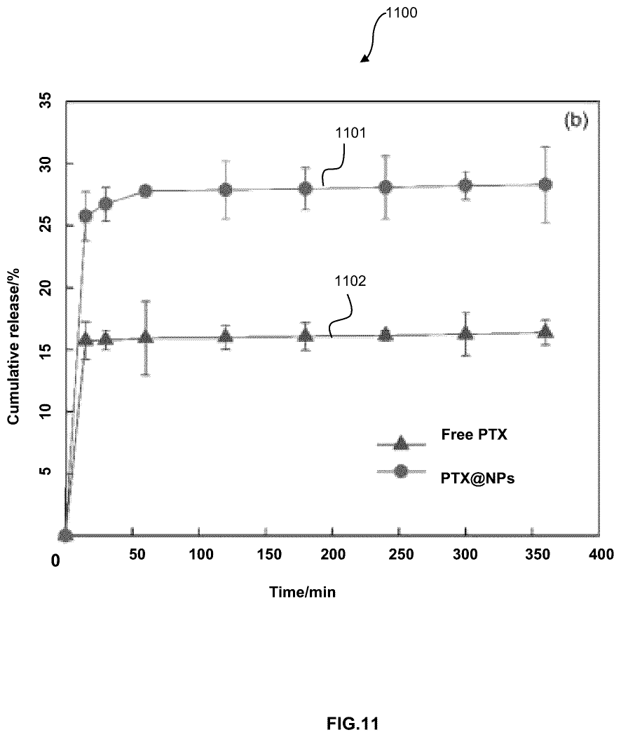

FIG. 11 shows the cumulative release of PTX from nano devices. The release behavior of PTX from nano devices in a graph 1102 was evaluated and compared to free PTX represented by a graph 1101 in the 1:9 solution of DMSO:PBS solution. As shown in graphs 1101 and 1102, at the early stage of the release, a large amount of PTX and free PTX were released from the particles, which were about 25% and 15%, respectively. These releases were obtained because of adsorbed PTX on the surface of nano devices. After that, PTX@NPs showed a steady rate of release that indicates the sustained increasing and long term release behavior of PTX@NPs. In contrast, a short-term release is obtained with free PTX which related to the high crystallinity of PTX. These results demonstrated that loading PTX into nano devices enhances its water-solubility and then increase its anticancer effectiveness.

### Cellular Uptake

The interaction between nano devices and plasma membranes determines cellular uptake, which is one of the most important processes governing the biological activity of nano devices. Multiple endocytic pathways facilitate the incorporation of nano devices into the cell. Upon nanoparticle exposure, the nonspecific adhesion of nanoparticles to cell membranes affects membrane morphology or permeability, and the resulting effects on the cytotoxicity test. Consequently, examining the interaction of nanoparticles with cell membranes is the first crucial step in comprehending biological responses. The ability of breast cancer cell line MCF7 to absorb nanoparticles was significantly greater than that of normal cell line L929. In cancer, genetic or environmental factors ignore signals that control cell division and cell death. Uncontrolled cell division causes fast cell development and localized malignancies. Tumor cells proliferate quickly, metastasize, and generate new blood arteries, leading to instability of the cellular membrane [27]. All these differences alter tumor cell interactions with applied nanostructures, can trigger cellular processes, and change cellular absorption, which is critical for targeted cancer therapy with nanoparticles. As a result, nanoparticles presented in MCF-7 breast cancer cells were significantly higher than in L929 fibroblast normal cells, further emphasizing the results obtained from the cytotoxicity experiments. Besides, the cellular uptake test was observed directly by the introduction of FITC-labeled nano devices, 4% of paraformaldehyde solution was used for fixing cells in the preparation of the specimen to evaluate cellular uptake by confocal microscopy. The green fluorescence particles were observed after 4 hours of incubation with FITC-labeled nano devices. This result showed that the prepared FITC-labeled nano devices internalized into MCF-7 cells were significantly higher than that of L929 cells. The nanoparticles were observed to be distributed inside cells, throughout the cytoplasm of the cells. It was previously confirmed that the nano devices show higher uptake in the cancer cell as compared to the normal cells [28]. As a result, it was evidence demonstrating that nano devices play an important role in the processing of drug delivery.

### Cytotoxicity

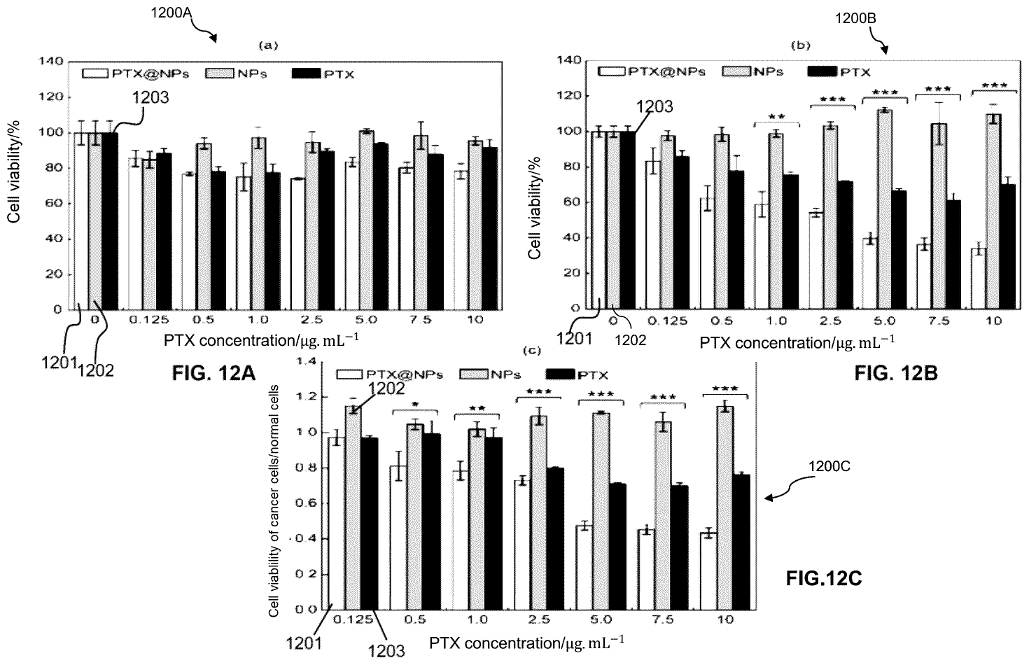

Finally referring to FIG. 12A-FIG. 12C, cell viability of nano devices is are shown. After characterizations, different concentrations of nano devices, PTX@NPs 1201, and PTX (0.1-10 μg·mL⁻¹ concentration of PTX) 1203 were used in the MTT assay. The cell viability test was investigated on a mouse fibroblast cell line (L929) to observe whether nano devices and PTX@ NPs 1201 are toxic to normal cell lines. As shown in FIG. 12A, under the tested concentrations, the samples had no considerable toxicity on the viability of L929 (cell viability >70%). In contrast, referring to FIG. 12B, when a breast cancer cell line (MCF-7) was treated with the samples, PTX@NPs treatment significantly decreased cancer cell viability at high concentrations as compared to PTX treatment (**p≤0.001, ***p≤0.005). It was confirmed that IC50 of PTX@NPs against MCF-7 was 2.29 μg·mL⁻¹, which was significantly lower than that of PTX treatment. The viability ratio of cancer cells (MCF-7)/normal cells (L929) was further calculated to evaluate the anticancer potential of PTX@NPs. Comparing the results of cell viability on cancer cells and normal cells showed that this ratio from PTX@NPs was significantly lower than that from PTX at high concentrations such as 0.5, 1, 2.5, 5, 7.5 and 10 μg·mL⁻¹ (*p≤0.05, **p≤0.01, ***p≤0.005) (FIG. 7c), suggesting that PTX@NPs improved the anticancer activity of PTX. The cellular morphology was observed from the microscopic DIC images. Many dead cancer cells with spherical shapes were observed when treated with PTX@NPs and were significantly higher than that of PTX treatment. Live/dead staining was further conducted with FDA and PI. Similar to the cytotoxicity results, these samples were barely toxic to normal cell line L929, in which less red fluorescent signals were observed. In contrast, PTX and PTX@NPs exhibited the capacity on causing death on cancer cell lines MCF7 expressed by less green-signals and more red-signals. However, PTX@NPs treatment exhibited less viable cells (green signal) than PTX treatment demonstrating their ability on the viability of cancer cells. These results demonstrated that improvement of PTX solubility by incorporating it into the nano devices significantly increased the anticancer efficacy. On the other hand, PTX@NPs showed low toxicity toward normal cells. These obtained results indicate the anticancer potential of PTX@NPs of the present invention.

### CONCLUSION

The present invention provides a new type of biodegradable disulfide-based PMO by incorporating disulfide linkers into the silica framework of the nano devices. In particular, the nano devices were characterized by SEM and TEM to show homogeneous spherical particles with small sizes, approximately 50 nm. In addition, the presence of disulfide in the structure of the nano devices was confirmed by XPS analysis. It can be noted that the utilization of biodegradable disulfide linkers in the structure contributed to fast degradation of the nano devices as complete degradation within 5 days. In this study, hydrophobic paclitaxel inhibitors were loaded into the porous organic structure of particles, with a high loading capacity up to 602 mg·g⁻¹. The release behavior with the steady release rate of PTX@NPs demonstrated the enhanced solubility of PTX. Besides, the cellular uptake studies also contributed that the nano devices can be internalized into malignant cells than in normal cells. Therefore, the results of MTT assays show the higher toxicity of PTX@NPs compared to free PTX in cancer cell line MCF-7 while the nano devices had no considerable toxicity on normal cells. These results demonstrated that the disulfide-based BPMO nano device is a promising nanocarrier to improve the clinical efficacy of hydrophobic drugs.

### INDUSTRIAL APPLICABILITY

The present invention provides a biodegradable periodic mesoporous organosilica nanomaterial with a small size of 50 nm to 100 nm, which is biodegradable in the human body with a size between 2 nm and 5 nm, making it to easily excrete naturally through the kidney and non-toxic to the body, able to respond to acidic pH to cleave chitosan components on the surface of the material. The method of synthesis of materials is feasible with low cost, using less toxic chemicals, short reaction time, and can be synthesized in large quantities on an industrial scale.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The flow diagrams depicted herein are just one example. There may be many variations to this diagram or the steps (or operations) described therein without departing from the spirit of the invention. For instance, the steps may be performed in a differing order or steps may be added, deleted or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should, therefore, be construed in accordance with the appended claims and any equivalents thereof.

### REFERENCES

[1] Q. Fu, D. Hargrove, X. Lu, Improving paclitaxel pharmacokinetics by using tumorspecific mesoporous silica nanoparticles with intraperitoneal delivery, Nanomed. Nanotechnol. Biol. Med. 12 (2016) 1951-1959.
[2] Y. Goto, N. Mizoshita, M. Waki, M. Ikai, Y. Maegawa, S. Inagaki, Synthesis and Applications of Periodic Mesoporous Organosilicas, Chemistry of Silica and ZeoliteBased Materials, Elsevier, 2019, pp. 1-25.
[3] R. Li, Y. Xie, Nanodrug delivery systems for targeting the endogenous tumor microenvironment and simultaneously overcoming multidrug resistance properties, J. Control. Release 251 (2017) 49-67.
[4] X. Guo, Y. Cheng, X. Zhao, Y. Luo, J. Chen, W.-E. Yuan, Advances in redoxresponsive drug delivery systems of tumor microenvironment, J. Nanobiotechnol. 16 (2018) 1-10.
[5] N. X. D. Mai, A. Birault, K. Matsumoto, H. K. T. Ta, S. G. Intasa-ard, K. Morrison, P. B. Thang, T. L. H. Doan, F. Tamanoi, Biodegradable periodic mesoporous organosilica (BPMO) loaded with daunorubicin: a promising nanoparticle-based anticancer drug, ChemMedChem 15 (2020) 593-599.
[6] J. Croissant, X. Cattoën, M. W. C. Man, A. Gallud, L. Raehm, P. Trens, M. Maynadier, J. O. Durand, Biodegradable ethylene-bis (Propyl) disulfide-based periodic mesoporous organosilica nanorods and nanospheres for efficient in-vitro drug delivery, Adv. Mater. 26 (2014) 6174-6180.
[7] L. Maggini, I. Cabrera, A. Ruiz-Carretero, E. A. Prasetyanto, E. Robinet, L. De Cola, Breakable mesoporous silica nanoparticles for targeted drug delivery, Nanoscale 8 (2016) 7240-7247, https://doi.org/10.1039/C5NR09112H.
[8] J. Croissant, Y. Fatieiev, K. Julfakyan, J. Lu, A. Emwas, D. H. Anjum, H. Omar, F. Tamanoi, J. Zink, N. M. Khashab, Biodegradable oxamide-phenylene-based mesoporous organosilica nanoparticles with unprecedented drug payloads for delivery in cells, Chem. Eur. J. 22 (42) (2016) 14806-14811.
[9] N. X. D. Mai, U.-C. N. Le, L. H. T. Nguyen, H. T. K. Ta, H. V. Nguyen, T. M. Le, T. B. Phan, L.-T. T. Nguyen, F. Tamanoi, T. L. H. Doan, Facile synthesis of biodegradable mesoporous functionalized-organosilica nanoparticles for enhancing the anticancer efficiency of cordycepin, Microporous Mesoporous Mater. 315 (2021), 110913.
[10] N. X. D. Mai, Y. T. Dang, H. K. T. Ta, J.-S. Bae, S. Park, B. T. Phan, F. Tamanoi, T. L. H. Doan, Reducing particle size of biodegradable nanomaterial for efficient curcumin loading, J. Mater. Sci. 56 (2021) 3713-3722.
[11] P. Damodaran, Mesoporous magnetite nanoclusters as efficient nanocarriers for paclitaxel delivery, ChemistrySelect 5 (2020) 9261-9268.
[12] J. Gao, K. Fan, Y. Jin, L. Zhao, Q. Wang, Y. Tang, H. Xu, Z. Liu, S. Wang, J. Lin, PEGylated lipid bilayer coated mesoporous silica nanoparticles co-delivery of paclitaxel and curcumin leads to increased tumor site drug accumulation and reduced tumor burden, Eur. J. Pharm. Sci. 140 (2019), 105070.
[13] L. Jia, J. Shen, Z. Li, D. Zhang, Q. Zhang, G. Liu, D. Zheng, X. Tian, In vitro and in vivo evaluation of paclitaxel-loaded mesoporous silica nanoparticles with three pore sizes, Int. J. Pharm. 445 (2013) 12-19.
[14] Y. He, S. Liang, M. Long, H. Xu, Mesoporous silica nanoparticles as potential carriers for enhanced drug solubility of paclitaxel, Mater. Sci. Eng. C 78 (2017) 12-17
[15] M. Najlah, A. Kadam, K.-W. Wan, W. Ahmed, K. M. Taylor, A. M. Elhissi, Novel paclitaxel formulations solubilized by parenteral nutrition nanoemulsions for application against glioma cell lines, Int. J. Pharm. 506 (2016) 102.
[16] L. Zhang, Y. Wang, Y. Yang, Y. Liu, S. Ruan, Q. Zhang, X. Tai, J. Chen, T. Xia, Y. Qiu, High tumor penetration of paclitaxel loaded pH sensitive cleavable liposomes by depletion of tumor collagen I in breast cancer, ACS Appl. Mater. Interfaces 7 (2015) 9691-9701.
[17] Z. Xu, S. Zhu, M. Wang, Y. Li, P. Shi, X. Huang, Delivery of paclitaxel using PEGylated graphene oxide as a nanocarrier, ACS Appl. Mater. Interfaces 7 (2015) 1355-1363.
[18] F. Danhier, P. Danhier, C. J. De Saedeleer, A.-C. Fruytier, N. Schleich, A. des Rieux, P. Sonveaux, B. Gallez, V. Pr′eat, Paclitaxel-loaded micelles enhance transvascular permeability and retention of nanomedicines in tumors, Int. J. Pharm. 479 (2015) 399-407.
[19] J. Hu, S. Fu, Q. Peng, Y. Han, J. Xie, N. Zan, Y. Chen, J. Fan, Paclitaxel-loaded polymeric nanoparticles combined with chronomodulated chemotherapy on lung cancer: in vitro and in vivo evaluation, Int. J. Pharm. 516 (2017) 313-322.
[20] X. Zheng, S. Feng, X. Wang, Z. Shi, Y. Mao, Q. Zhao, S. Wang, MSNCs and MgOMSNCs as drug delivery systems to control the adsorption kinetics and release rate of indometacin, Asian J. Pharm. Sci. 14 (2019) 275-286.
[21] Z. Zhao, Y. Gao, C. Wu, Y. Hao, Y. Zhao, J. Xu, Development of novel core-shell dual-mesoporous silica nanoparticles for the production of high bioavailable controlled-release fenofibrate tablets, Drug Dev. Ind. Pharm. 42 (2016) 199-208.
[22] D. Esquivel, O. van den Berg, F. J. Romero-Salguero, F. Du Prez, P. Van Der Voort, 100% thiol-functionalized ethylene PMOs prepared by “thiol acid-ene” chemistry, Chem. Commun. 49 (2013) 2344-2346.
Mulder, High-performance and low-cost sodium-ion anode based on a facile black phosphorus-carbon nanocomposite, ChemElectroChem 4 (2017) 2140-2144.
[24] J. V. Rojas, M. Toro-Gonzalez, M. C. Molina-Higgins, C. E. Castano, Facile radiolytic synthesis of ruthenium nanoparticles on graphene oxide and carbon nanotubes, Mater. Sci. Eng. B 205 (2016) 28-35.
[25] N. X. D. Mai, T.-H. T. Nguyen, L. B. Vong, M.-H. D. Dang, T. T. T. Nguyen, L. H. T. Nguyen, H. K. T. Ta, T.-H. Nguyen, T. B. Phan, T. L. H. Doan, Tailoring chemical compositions of biodegradable mesoporous organosilica nanoparticles for controlled slow release of chemotherapeutic drug, Mater. Sci. Eng. C 127 (2021), 112232,
[26] R. Fang, S. Yang, Y. Wang, H. Qian, Nanoscale drug delivery systems: a current review on the promising paclitaxel formulations for future cancer therapy, Nano 10 (2015), 1530004.
[27] S. C. Gupta, D. Hevia, S. Patchva, B. Park, W. Koh, B. B. Aggarwal, Upsides and downsides of reactive oxygen species for cancer: the roles of reactive oxygen species in tumorigenesis, prevention, and therapy, Antioxid. Redox Signal. 16 (2012) 1295-1322.
[28] L. B. Vong, S. Kimura, Y. Nagasaki, Nowty designed silica-containing redox nanoparticles for oral delivery of novel TOP2 catalytic inhibitor for treating colon cancer, Adv. Healthc. Mater. 6 (2017), 1700428.

1. A nano device, comprising:
a biodegradable periodic mesoporous organosilicas (BPMO) framework comprising a structure formed by Si—O—Si covalent bonds and a plurality of mesopores configured to store drug cargos therein by means of disulfide covalent bonds; and
an independent and separate chitosan layer, deposited directly on said framework, operable to coat and protect said framework layer from leaking said drug cargos in a first condition and to degrade so as to expose said drug cargos in a second condition, and wherein in said second condition said framework releases said drug cargos from said plurality of mesopores by allowing a predetermined concentration of glutathione to break said disulfide (S—S) covalent bonds.

2. The nano device of claim 1 wherein said first condition is a pH of 7.4.

3. The nano device of claim 1 wherein said second condition is a pH of 4.0-5.5.

4. The nano device of claim 1 wherein said predetermined concentration of glutathione is 10 millimoles (mM).

5. The nano device of claim 1 wherein said separate and independent chitosan layer has molecular weight between 50 kDa to 300 kDa.

6. The nano device of claim 1 wherein said framework has a spherical shape with a diameter between 50 nm to 100 nm.

7. The nano device of claim 1, wherein said plurality of mesopores each has a diameter between 1 nm to 3 nm.

8. The nano device of claim 1 is characterized by having a spherical shape with a specific surface area between 150 m²/g to 900 m²/g.

9. The nano device of claim 1 wherein said structure is formed by a reaction between alkyl alkoxy silane or aryl alkoxy silane and sulfur alkoxy silane.

10. The nano device of claim 9 wherein said sulfur alkoxy silane is selected from bis[3-(triethoxysilyl)propyl]tetrasulfide and bis[3-(triethoxysilyl)propyl]disulfide.

11. The nano device of claim 1 wherein said alkyl alkoxy silane or aryl alkoxy silane and sulfur alkoxy silane further comprise disulfide (S—S) or tetrasulfide (S—S—S—S) bridges.

12. The nano device of claim 11 wherein said alkyl alkoxy silane or aryl alkoxy silane is selected from 1,2-Bis(triethoxysilyl)ethane, 1,2-Bis(triethoxysilyl)ethylene, 1,4-Bis(triethoxysilyl)benzene and 4,4′-Bis(triethoxysilyl)biphenyl.

13. The nano device of claim 1 wherein said BPMO framework has a drug loading capacity between 300 mg/g to 1000 mg/g.

14. A method of synthesizing a drug delivering nano device, comprising:
i) synthesizing a biodegradable periodic mesopores organosilicas (BPMO) framework having a structure formed by Si—O—Si covalent bonds and a plurality of mesopores configured to store drug cargos therein by means of disulfide (S—S) covalent bonds; and
ii) coating said framework with a separate and independent layer of chitosan by depositing said chitosan layer directly on said BPMO framework; wherein said chitosan layer is operable to cover and protect said BPMO framework layer from leaking said drug cargos in a first condition and to degrade so as to expose said drug cargos in a second condition, and wherein said BPMO framework releases said drug cargos by allowing a predetermined concentration of glutathione to break said disulfide (S—S) covalent bonds.

15. The method of claim 14 further comprising adding drug cargos into said pores by mixing said drug cargos with said BPMO framework by mixing prior to said coating step (b).

16. The method of claim 14 wherein said step (b) further comprises:
(i) stirring said chitosan layer and said BPMO framework together for 48 hours at room temperature; and
(ii) centrifuging and washing said stirred and washed said BPMO framework with ethanol, water, and ethanol; wherein centrifugation for 15 minutes to 45 minutes with a speed of 14,000 rpm to 20,000 rpm.

17. The method of claim 14 wherein said step (a) further comprising:
(i) mixing an alkaline solution with a surfactant to form a catalyst solution;
(ii) adding dropwise silane precursors first into said catalyst solution and followed by sulfur alkoxy silane; and
(iii) condensing a product of step (ii) for time for 2 hours at 80° C. to collect said framework.

18. The method of claim 14, wherein a weight ratio between surfactant and alkaline solution is in the range from 3:1 to 9:1; a weight ratio between said surfactant to alkyl alkoxy silane or aryl alkoxy silane is in the range from 2:1 to 3:1; and a weight ratio between said surfactant to said sulfur alkoxy silane is in the range from 1:1 to 1:1.5.

19. A method for curing cancers using a drug delivering nano device, comprising:
(a) a biodegradable periodic mesopores organosilicas (BPMO) framework comprising a structure formed by Si—O—Si covalent bonds and a plurality of mesopores configured to store drug cargos therein by disulfide covalent bonds;
(b) adding drug cargos into said plurality of pores by mixing said drug cargos with said (BPMO) framework;
(c) depositing an independent and separate chitosan layer directly on said (BPMO) framework, wherein said chitosan layer is operable to coat and protect said (BPMO) framework layer from leaking said drug cargos in a first condition and to degrade so as to expose said drug cargos in a second condition, and wherein said framework releases said drug cargos by allowing a predetermined concentration of glutathione to break said disulfide (S—S) covalent bonds; and
(d) injecting said drug delivery nano devices into victims who suffer from said cancers.

20. The method of claim 19 wherein said first condition is a pH of 7.4, said second condition is a pH of 4.0-5.5, and said predetermined concentration of glutathione is 10 millimoles (mM).
